(19) United States
(12) Patent Application Publication
(10) Pub. No.: US 2023/0263473 A1
(43) Pub. Date: Aug. 24, 2023

(54) BIOMEDICAL BIG DATA ANALYSIS PROGRAM

(71) Applicant: UNIVERSITY OF CONNECTICUT, FARMINGTON, CT (US)

(72) Inventors: Beiyan Zhou, Avon, CT (US); Chuan Li, Schijiazhuang, Hebei (CN)

(21) Appl. No.: 18/017,551

(22) PCT Filed: Jul. 22, 2021
(86) PCT No.: PCT/US21/42787

§ 371 Date: Jan. 23, 2023

Related U.S. Application Data
(60) Provisional application No. 63/055,234, filed on Jul. 22, 2020.

(51) Int. Cl. A61B 5/00; G16H 50/20

(52) U.S. Cl. CPC A61B 5/72; G16H 50/20

(57) ABSTRACT

Disclosed herein is a biomedical data analysis program (“MUSTER”) that facilitates extraction of key features from big data sets and can be leveraged for different discovery-oriented goals and purposes. In particular, the disclosed biomedical data analysis program is used for mutation-survival interrogation and provides an evolutionary probability distribution for gene signature identification.

### CROSS-REFERENCE TO RELATED APPLICATION

This application claims priority to U.S. Provisional Application No. 63/055,234, filed Jul. 22, 2020, which is hereby incorporated by reference in its entirety.

### BACKGROUND

Gene expression signatures are powerful tools that can reveal a range of biologically and clinically important characteristics of biological samples. In recent years, gene signatures have been developed that can differentiate distinct subtypes of tumors, identify important cellular responses to their environment (hypoxia), predict clinical outcomes in cancer, and model the activation of signaling pathways (West M, Ginsburg GS, Huang AT, Nevins JR: Embracing the complexity of genomic data for personalized medicine. Genome Res 2006, 16(5):559-66). The power of gene expression signatures derives from their ability to connect an in vitro experimental state with an in vivo one in a quantitative manner. Three major obstacles hinder the broad use of gene signatures. First, gene expression signature analysis requires the rigorous application of complex statistical methodologies on gene expression data. Second, it requires the acquisition and validation of data that properly capture the biological state of interest. Third, it requires a computational infrastructure that makes available the statistical software and data in an easy-to-use interface. Accordingly, improved methods are needed in the art to broaden the use of gene signatures and overcome aforesaid obstacles.

### SUMMARY

Disclosed herein is a biomedical data analysis program (“MUSTER”) that facilitates extraction of key features from big data sets and can be leveraged for different discovery-oriented goals and purposes. In particular, the disclosed biomedical data analysis program is used for mutation-survival interrogation and provides an evolutionary probability distribution for gene signature identification.

Accordingly, in one aspect, disclosed herein are methods of generating a feature set, based on biological signature data, used for predicting an outcome, the method comprising:
generating a representation of a mutated species from a representation of an initial species comprising biological signature data;
comparing a measured performance of the representation of the mutated species with a measured performance of the representation of the initial species based on associated outcome data;
selecting one of the representation of the mutated species and the representation of the initial species based on the results of the comparing to serve as the representation of the initial species in a next iteration of the generating and comparing; and
outputting a representation of a super species based on a final mutated species, the representation of the super species comprising a set of biological signature features predictive of the outcome.

In some embodiments of methods of generating a feature set, the measured performance is an area under a receiver operating characteristic curve determined from at least one classification threshold.

In some embodiments of methods of generating a feature set, the biological signature data comprises proteomic data, bulk transcriptome data, single-cell transcriptome data, genomic data, metabolomic data, microbiotomic data, or any combination thereof.

In some embodiments of methods of generating a feature set, the super species comprises a set of genes associated with resistance to a treatment for acute myeloid leukemia (AML).

In some embodiments of methods of generating a feature set, the super species comprises a set of genes associated with cardiovascular disease risk.

In some embodiments of methods of generating a feature set, the super species comprises a set of genes associated with cancer relapse.

In some embodiments of methods of generating a feature set, the super species comprises a set of genes associated with post-traumatic stress disorder (PTSD).

In some embodiments of methods of generating a feature set, the method further comprises using the set of biological signature features to determine an outcome.

Also provided herein is a method of predicting an outcome comprising determining an outcome for test biological signature data based on the super species output determined in the methods of generating a feature set disclosed herein.

In another aspect, provided herein are methods, comprising:
Receiving a biological data set;
Extracting an identification (ID) for each entry of the biological data set a set of IDs;
Generating an initial data set from the biological data set, wherein the initial data set is a subset of the biological data set;
Generating a mutated data set from the initial data set;
Measuring performance of the mutated data set and the initial data set; and
Selecting one of the mutated data set or the initial data set to create an output set based the performance of each respective data set.

In some embodiments of these methods, the performance is based on an area under a curve of a model data set.

In some embodiments of these methods, the biological data set comprises transcriptiomics from RNA sequence data.

In some embodiments of these methods, the biological data set comprises single cell transcriptomics.

In some embodiments of these methods, biological data set comprises proteomics.

In some embodiments of these methods, the biological data set comprises single nucleotide polymorphism genomic data.

In some embodiments of these methods, measuring performance of the mutated data set and the initial data set comprises propagating the mutated data set and the initial data set in parallel multiple times.

In some embodiments of these methods, the output set predicts an outcome of the biological data set.

### BRIEF DESCRIPTION OF THE FIGURES

FIG. 1 is a flowchart illustrating elements of an example embodiment of a computer-implemented method, titled the MUSTER program.

FIG. 2 is a flowchart illustrating elements of an example module, titled the EVOVE module, of a MUSTER program.

FIG. 3 is a flowchart illustrating elements of an example implementation of the MUSTER program, titled MUSTER-RNA-seq.

FIG. 4 is a flowchart illustrating elements of an example implementation of the MUSTER program, titled MUSTER-Single Cell Transcriptomics.

FIG. 5 is a flowchart illustrating elements of an example implementation of the MUSTER program, titled MUSTER-Proteomics.

FIG. 6 is a flowchart illustrating elements of an example implementation of the MUSTER program, titled MUSTER-Genomics (SNPs).

FIG. 7 shows an efficacy comparison for the prediction model AUC by the MUSTER program as compared to a known report.

FIG. 8 shows an efficacy comparison for predicting cardiovascular events by MUSTER-Red-RNAseq program as compared to the Framingham and JAMA scores.

FIG. 9 shows an efficacy comparison for predicting cardiovascular events by MUSTER-Red-Proteomics program as compared to the Framingham and JAMA scores.

FIG. 10 is a flowchart illustrating elements of an example implementation of the MUSTER program, titled MUSTER-Pink-RNA.

FIG. 11 is a flowchart illustrating elements of an example implementation of the MUSTER program, titled MUSTER-Pink-SNPs.

FIG. 12 shows the MUSTER-Pink-RNA prediction model efficacy assessment from the MUSTER core program.

FIG. 13 shows the MUSTER-Pink-SNPs prediction model efficacy assessment from the MUSTER core program.

FIG. 14 shows the MUSTER-Pink-RNA breast cancer relapse score prediction model using gene expressing levels in breast cancer patients with or without relapse.

FIG. 15 shows the MUSTER-Pink-SNPs breast cancer relapse score prediction model using gene expressing levels in breast cancer patients with or without relapse.

FIG. 16 is a flowchart illustrating elements of an example implementation of the MUSTER program, titled MUSTER-Red-RNA.

FIG. 17 is a flowchart illustrating elements of an example implementation of the MUSTER program, titled MUSTER-Red-Protein.

FIG. 18 shows the MUSTER-Red-RNA prediction model efficacy assessment from the MUSTER core program.

FIG. 19 shows the MUSTER-Red-Protein prediction model efficacy assessment from the MUSTER core program.

FIG. 20 shows the MUSTER-Red-RNA cardiovascular risk score prediction model using gene expression levels in blood cells independent of lipid profiles.

FIG. 21 shows the MUSTER-Red-Protein cardiovascular risk score prediction model using gene expression levels in blood cells independent of lipid profiles.

FIGS. 22A-22B show the efficacy comparison of the prediction model for MUSTER-Red-RNA (FIG. 22A) and MUSTER-Red-RNA (FIG. 22B) as compared to other tradition cardiovascular risk score systems.

FIG. 23 shows a comparison of MUSTER-Red-RNA as compared to other risk score models.

FIG. 24 shows a comparison of MUSTER-Red-Protein as compared to other risk score models.

FIG. 25 is a flow chart showing MUSTER-AML's signature gene identification and response prediction of AML resistance.

FIG. 26 is a flowchart illustrating elements of an example implementation of the MUSTER program, titled MUSTER-AML.

FIG. 27 shows the MUSTER-AML prediction model efficacy assessment from the MUSTER core program.

FIG. 28 shows the accuracy of MUSTER-AML in dataset testing.

### DETAILED DESCRIPTION

### Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, “optional” or “optionally” means that the subsequently described event or circumstance may or may not occur, and that the description includes instances where said event or circumstance occurs and instances where it does not.

MUSTER Program

Disclosed herein is a program, “MUSTER,” that facilitates extraction of key features from big data sets and can be leveraged for different discovery-oriented goals and/or purposes. In some embodiments, an “Ancestor” seed list can be produced from a random subset of IDs from raw data that does not rely on preset assumptions. MUSTER then can randomly introduce “mutations” into the existing list to test the impact on the model efficacy. This subsequently can undergo a competition/survival process.

MUSTER is built on several core algorithms with strong capacity to introduce “mutation factors” (Mutator Node), followed by a sanity size check procedure (Size Keeper Node), and a new outcome (intermediate species) competition-survival test (Competition Node Selection Node). The introduced mutation in each round is assigned through a probability function (Random Probability Distribution, RPD rate) algorithm. The organic arrangement of these key features in MUSTER is strengthened through a stringent competition-survival design to ensure both efficacy, unbiased discovery as well as precision in biomedical applications. In addition, the nature of the disclosed program is highly tolerant to the input data format, and thereby allows for successful processing of various big data sets for biomedical applications.

In contrast, other current technologies use similarity comparison or differential level comparison to extract gene IDs or SNPs for association for big data analyses. Given the complex biomedical background and multiple contributing factors, these programs often build upon a strong data trimming bias and thereby reduce the novel signature identification and efficacy of the prediction or diagnostic models. Other data processing processes that employ established mathematical models rely strongly on established functions on the basis of the presumption of each study to avoid deviation of the biological context from the mathematical formula; however, this often causes such processes to lose the targeted precision needed for biomedical applications.

MUSTER provides users with several advantages as compared to existing systems. Such advantages include, but are not limited to: a flexible input capacity; an unbiased process that enables a novel signature for feature discovery; a high precision for medical discovery; and potent application capacity.

MUSTER can process a variety of big data format that were generated using different technologies, including protein profiles (proteomics), bulk transcriptomics (conventional RNA-seq data sets generated from either a mixture of cell types or tissues), single-cell transcriptomics (transcriptomics at single cell level), genomics (genomic DNA profiles that containing either inherited or acquired mutations or single-nucleotide polymorphism), or other high-through put profiles (such as metabolomics, microbiotomics, etc). Therefore, in some embodiments provided herein, the computer-implemented method can accept flexible input data formats, including transcriptiomics from RNA-seq data, single cell transcriptomics, proteomics (protein detection profiles), and SNP genomic data.

MUSTER is also enabled through the incorporation of several novel algorithms to initiate the process with a random subset of IDs (“the ancestor”) that does not rely on preset assumptions for the investigation. MUSTER is also enabled, for each run, to randomly introduce mutations (both identity of new IDs and the quantity of mutated IDs) into the existing list (the ancestor for the first run, and the “old species” for the later runs >2) and test their impact on the modeling efficacy. Therefore, in some embodiments, provided herein MUSTER provides for bias reduction.

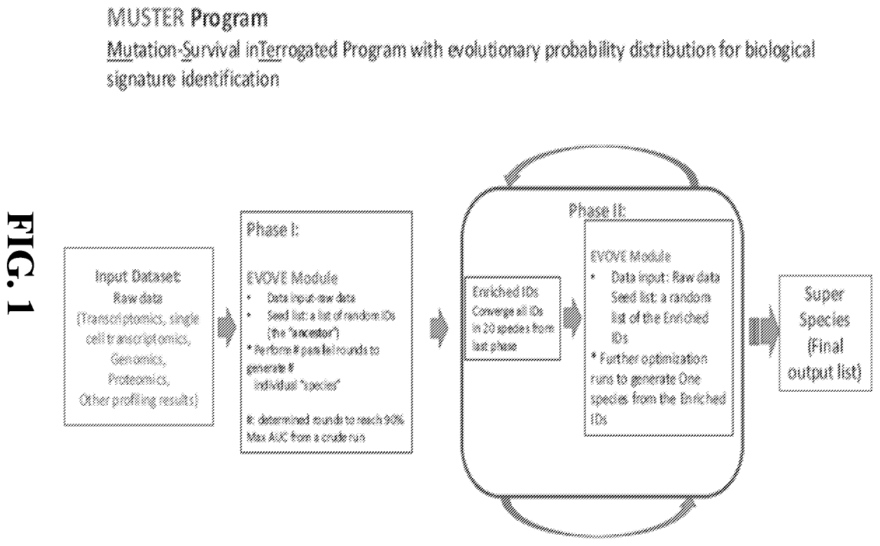

MUSTER implements a two-phased process (FIG. 1) to organically incorporate several innovative algorithms for big data mining. The quality and signature size check performed in each round with dynamic introduced mutations (both in the mutated IDs and quantity of mutations) into the existing gene list are subsequently evaluated with Competition and Survival process to select the ones with better detection efficacy for each project goal. The number of cycled-runs are determined using a monitored efficacy theory to ensure the best final output (super species). Non-contributing IDs in the list are each examined to ensure the precision in model calculation in each run. Therefore, in some embodiments provided herein is a computer-implemented method that applies a two-phased procedure to generate a final feature set.

As shown in FIG. 1, MUSTER is executed in two phases. Phase I utilizes an EVOVE module. The EVOVE module uses input-raw data. The input-raw data is used to: a) generate a seed list that consisting a list of random IDs from the raw data (the ancestor); b) the total number of program cycles needed to reach the 90% of estimated max AUC; and c) perform a lines of parallel rounds independently to generate multiple individual lists (species).

In Phase II, a new pool of all IDs from the Phase I list (species) is used to generate a concentrated candidate pool for “secondary ancestor” randomized selection. EVOVE module is executed in cycles using the enriched pool of IDs and tested with monitored AUC output for efficacy of the modeling.

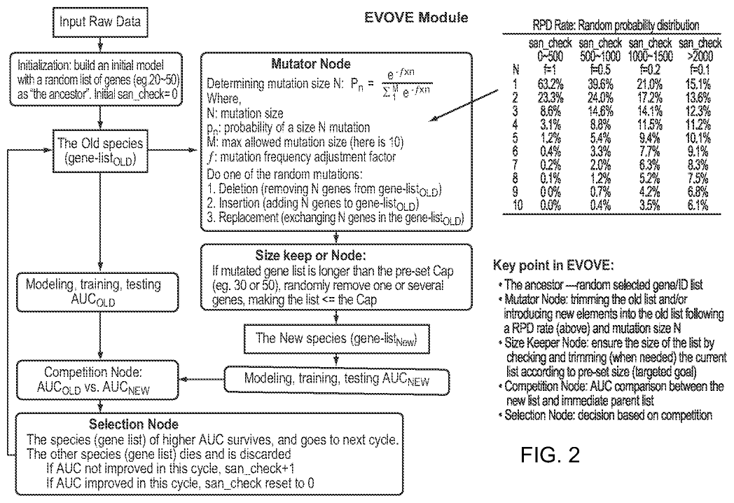

The MUSTER was built upon several crucial and innovative algorithms. In particular, as shown in FIG. 2, the random initiating list; the potent EVOVE module; the mutation-survival stage; and the gene list.

The random initiation list avoids the potential bias that can build up, thus allow novel feature identification.

The potent EVOVE module is the core algorithm in MUSTER. EVOVE comprises four nodes: the Mutator Node, the Size Keeper Node, the Competition Node, and the Selection Node (Survival Node).

The mutation-survival stage is enabled by two phases: introducing mutation(s) into the existing list (the old species, also termed gene-list_{OLD}) at a RPD rate (a stage-wide random probability distribution function with different frequency (0 value for each stage). The mutation size N is calculated using a new function.

Efficacy of the gene list-based modeling is monitored in each round through a competition and selection node to ensure time saving and precision of the overall procedure.

The final output of key signature features for each analysis provides a list of IDs most pertinent to the project goal, including biomarker identification, disease risk factor identification, cancer re-occurrence risks, or treatment efficacy prediction and others. Therefore, in some embodiments, the generated final feature set ultimately can be for disease diagnosis (e.g., biomarker identification), contributing feature identification (e.g., signature gene set characterization for a specific disease), and/or feeding into disease prediction models (e.g., identify core gene sets for treatment response and/or risk of cancer reoccurrence).

MUSTER Applications

In some embodiments, the method is applied to different projects and/or goals, including, but not limited to, biomarker identification, disease risk factor identification, cancer re-occurrence risks, treatment efficacy, and finance.

Cardiovascular Risk

While there are existing cardiovascular risk assessments used in clinical practice, these systems have limited clinical measurements (e.g., body weight, blood pressure, etc.) and heavily rely on lipid profiles (e.g., HDL and LDL cholesterol levels). Several weaknesses shared by these models include: accuracy; and application to predict cardiovascular risk in those currently under lipid control regimens.

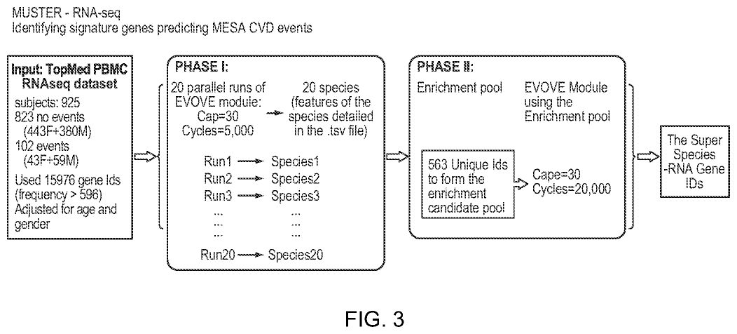

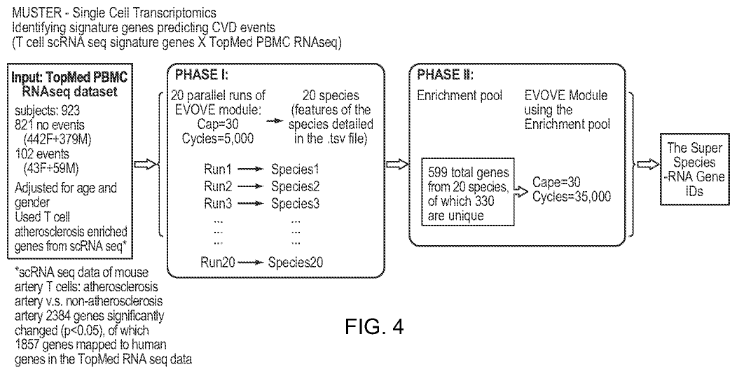

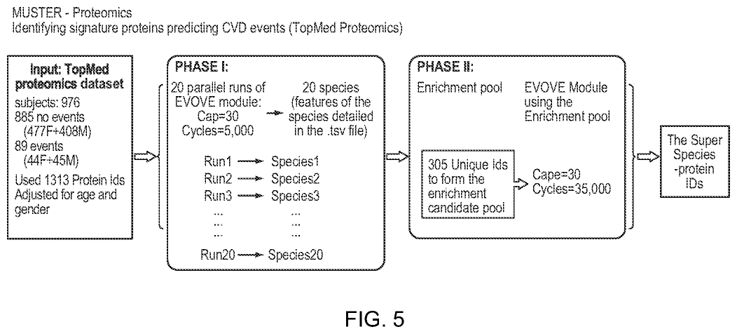

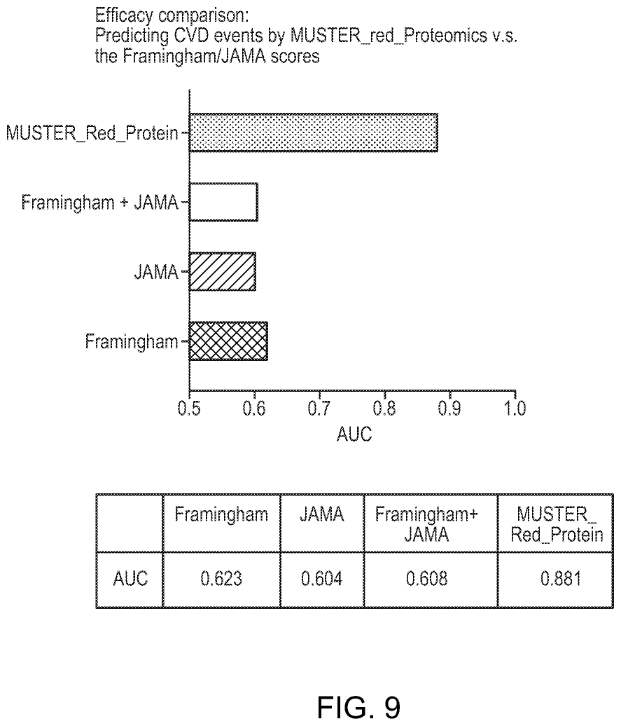

MUSTER is used to identify cardiovascular event risks (FIG. 3-FIG. 5, and FIG. 9). For example, MUSTER can identify risk gene sets for cardiovascular event risks in general human populations using single cell transcriptomics (MUSTER sc) (FIG. 3). In other embodiments, MUSTER can be used for bulk RNA transcriptomics (MUSTER brRed) (FIG. 4). In still other embodiments, MUSTER is used for proteomics (MUSTER_proRed) (FIG. 5).

In particular, MUSTER-Red can be applied for the prediction of health risks of a cardiovascular event independent of a lipid controlling treatment. Two sub-programs provide a set of risk gene lists that can be measured at either RNA (RNA-seq, quantitative PCR, microarray, or single-cell RNA-seq) or protein (ELISA measurement, proteomics profiling or other protein deductions methods) levels.

Assessing a subject's risk for cardiovascular events can be useful for the prevention, treatment, diagnosis, or insurance condition assessment. The provided gene lists also can be used as molecule targets for drug design and development.

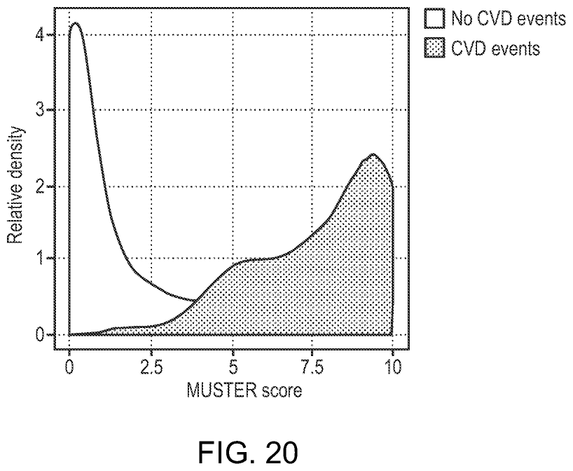

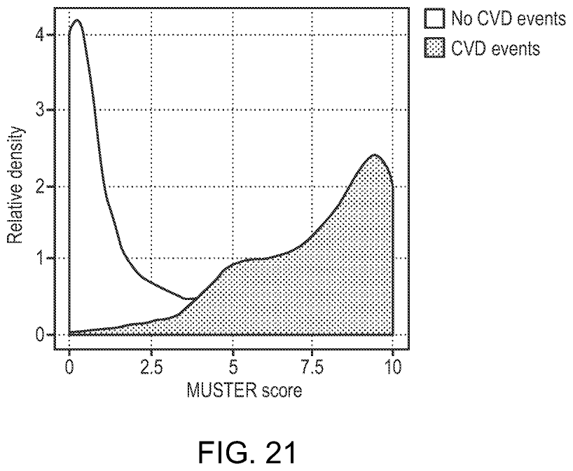

MUSTER-Red is an innovative program that does not rely on limited physiological measurements (which often largely fluctuate). MUSTER-Red also can provide a risk assessment in subjects that are already on a lipid controlling treatment (FIG. 20 and FIG. 21).

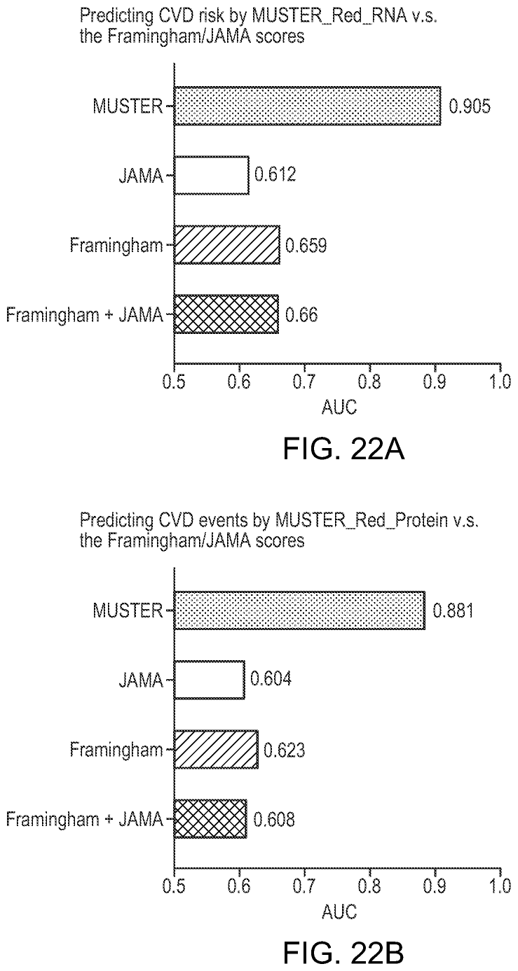

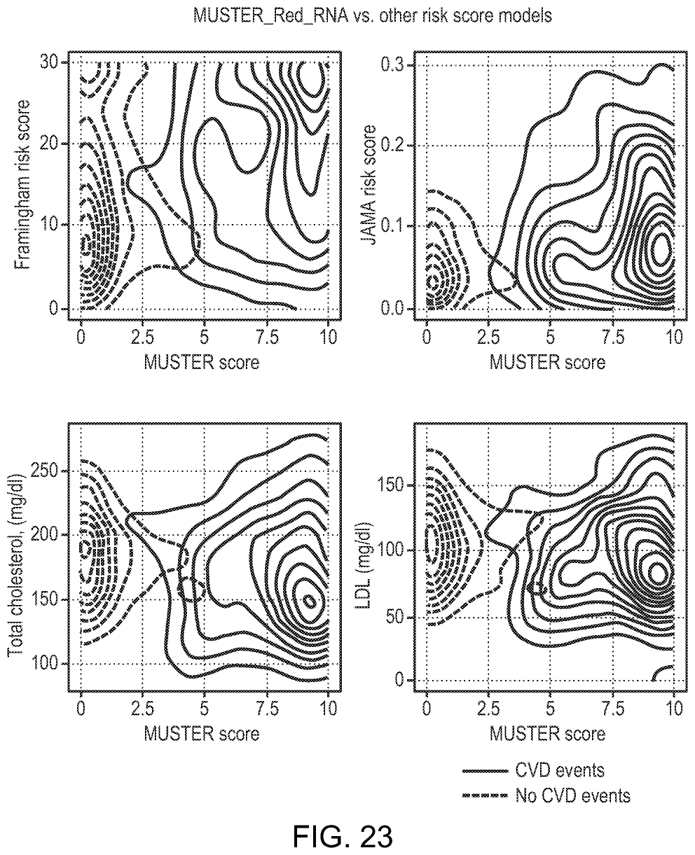

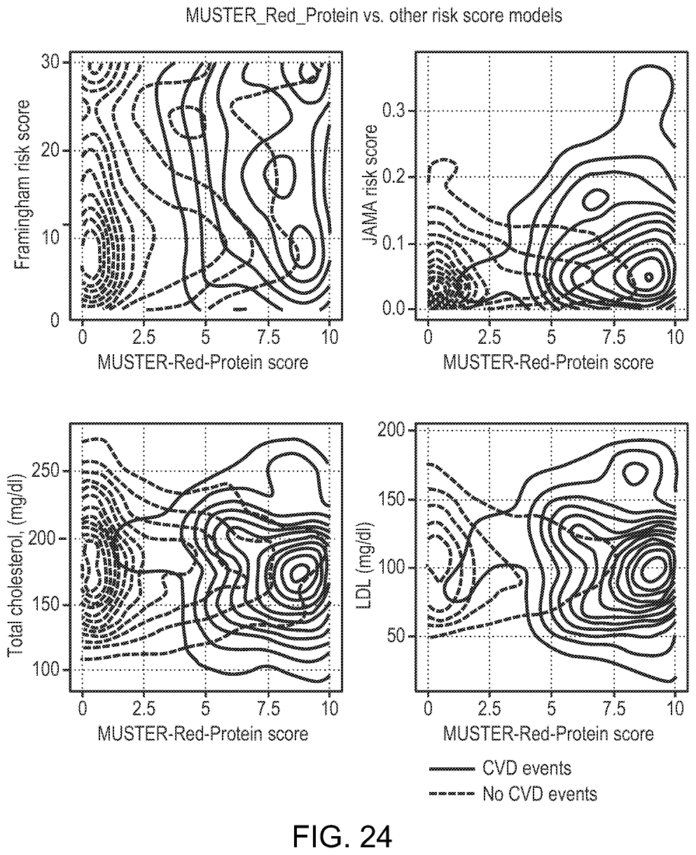

One major advantage of MUSTER-Red-RNA and MUSTER-RED-Protein is that it can provide a more accurate assessment of a cardiovascular risk in subjects compared to existing cardiovascular risk assessments that are currently in use in clinical practice (FIGS. 22-24). Application of these two models can be used directly in patient blood samples and thus can save time and provide a non-invasive sample collection protocol.

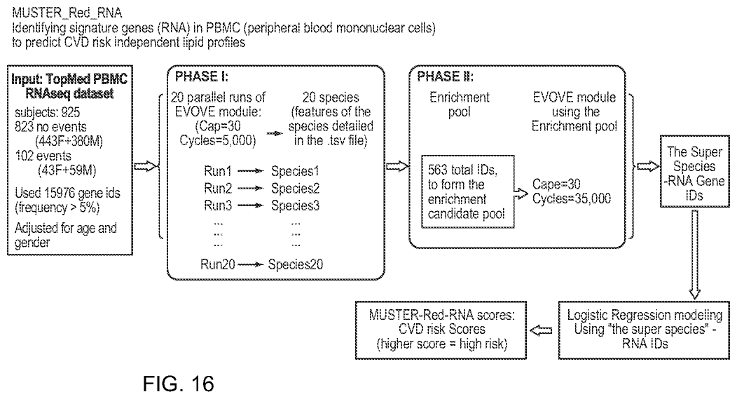

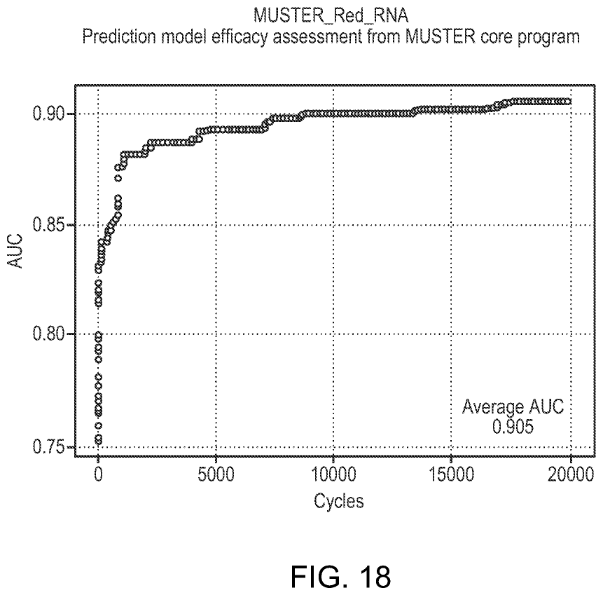

MUSTER-Red-RNA (FIG. 16) uses a set of 30 genes (Table 1) for assessment of cardiovascular risk using peripheral blood sample profiles. The prediction model using the TopMED dataset (923 profiles, PBMC RNA-seq) achieved an efficacy of AUC 0.905 (FIG. 18).

TABLE 1
30 Genes for MUSTER-Red-RNA

| | | |
|---|---|---|
| RP11_424G14.1, | B2M, | RALA, |
| RP3_508I15.9, | COL6A2, | GPR25, |
| TRAV34, | COL9A2, | PGS1, |
| IFI27L1, | MRVI1, | DDX17, |
| GPR75, | CRACR2B, | SCYL3, |
| PPDPF, | GPX7, | RP11_115C21.2, |
| SNORA10, | PINX1, | MTND2P28, |
| IGSF8, | SASS6, | DLEU2_1, |
| DHRSX, | AC006116.27, | TMEM144, |
| PPA2, | DDX31, | FCGR1B |

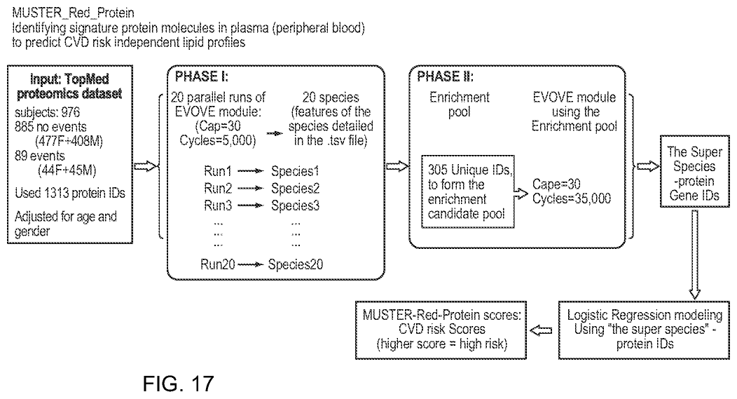

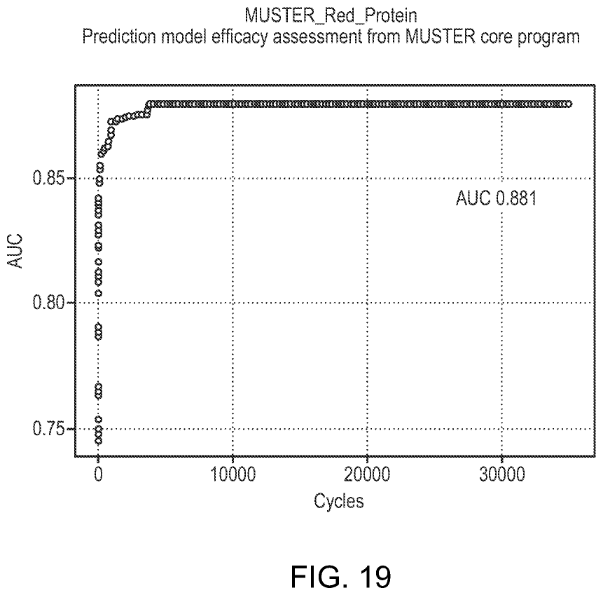

MUSTER-Red-Protein (FIG. 17) uses a set of 30 protein genes (Table 2) for assessment of cardiovascular risk using human blood samples. The prediction model achieved AUC of 0.881 (FIG. 19) in the TopMED dataset that contained a total of 976 profiles.

TABLE 2
30 genes for MUSTER -Red-Protein.

| | | |
|---|---|---|
| MMP12, | MDM2, | HCE004333, |
| SIGLEC6, | HAVCR2, | TNNI2, |
| P4HB, | IL18BP, | BPI, |
| ADAM9, | LTBP4, | LMNB1, |
| NANOG, | FCN3, | FN1, |
| KLK14, | HMGB1, | TFF1, |
| SFTPO, | CHEK2, | PLA2G1B, |
| LGALS7, | CXCL8, | TNFRSF10D, |
| CCL3L1, | HAT1, | ACVRL1, |
| CA9, | IL25, | EGFR.1 |

Breast Cancer Relapse

To date, there is no effective model to predict cancer relapses in patients who have had surgery. Regardless of clinical stage, over 15% of patients develop recurrence. To develop precision treatment for these patients, there is an urgent and unmet demand to identify cancer relapse risk. Cancer relapse risk assessment in this context therefore is underdeveloped and can directly affect the survival rate of cancer patients, their long-term quality of life, and overall incurred cost of treatment.

Using MUSTER, a recurrence risk assessment model was developed to provide strong efficacy and enable enhanced precision medicine for predicting cancer relapses, particularly for breast cancer.

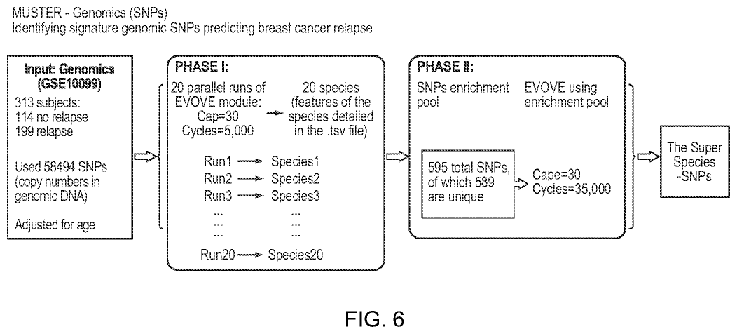

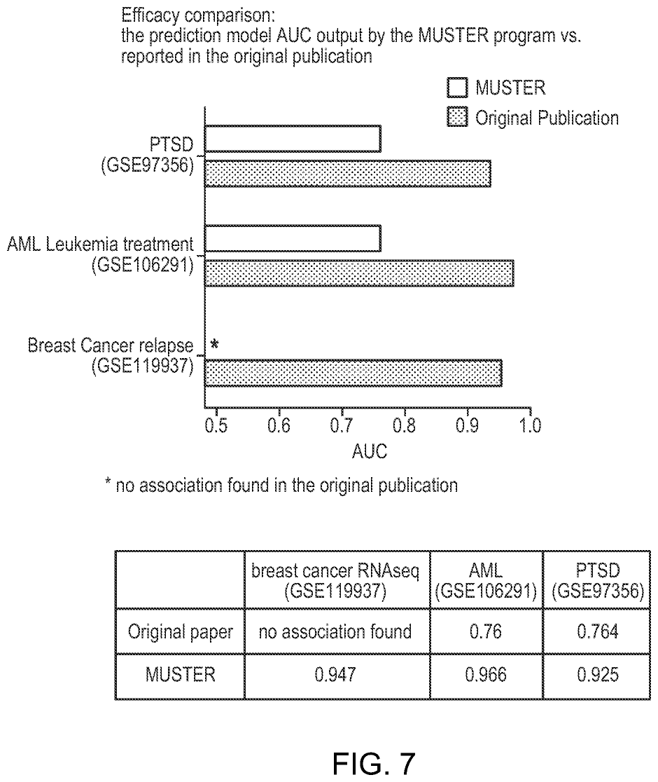

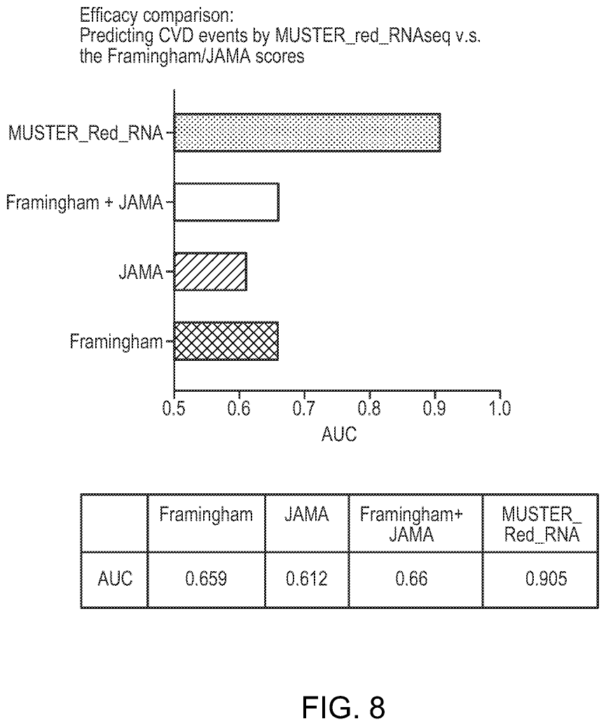

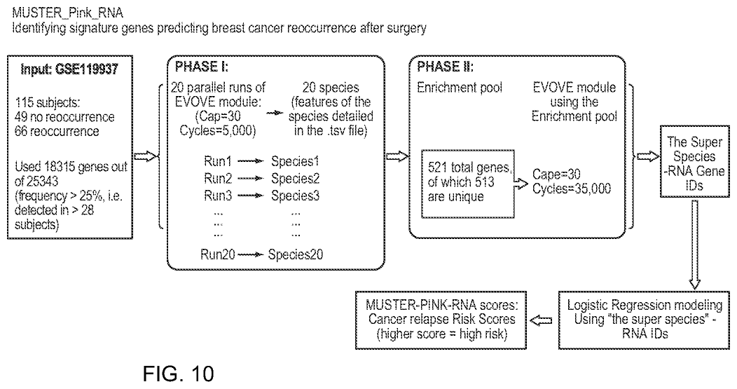

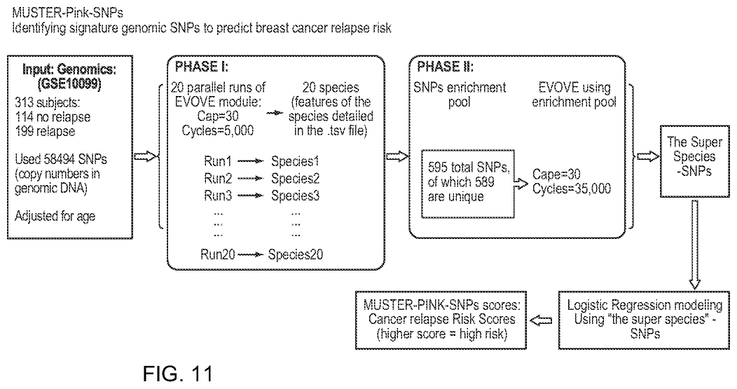

In some embodiments, MUSTER is used predict risk for breast cancer relapse (FIG. 6 and FIG. 7). In particular, MUSTER is used predict risk for breast cancer relapse with SNPs profiles (MUSTER-SNP_Pink) (FIG. 11) and bulk RNA-seq transcriptomics (MUSTER_brPink) (FIG. 10).

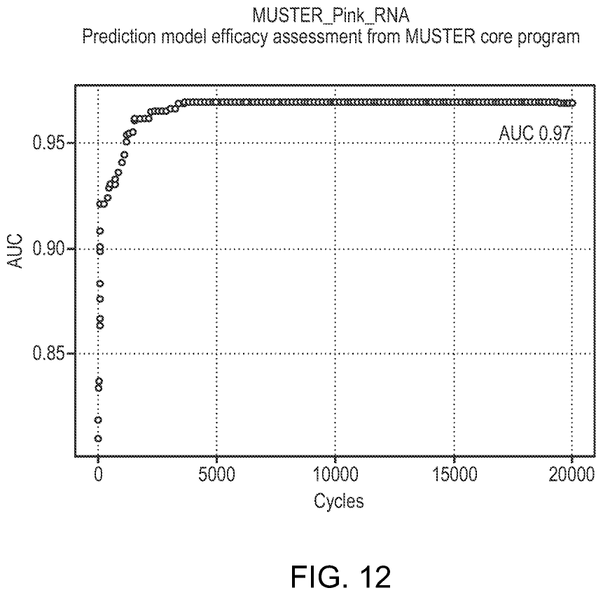

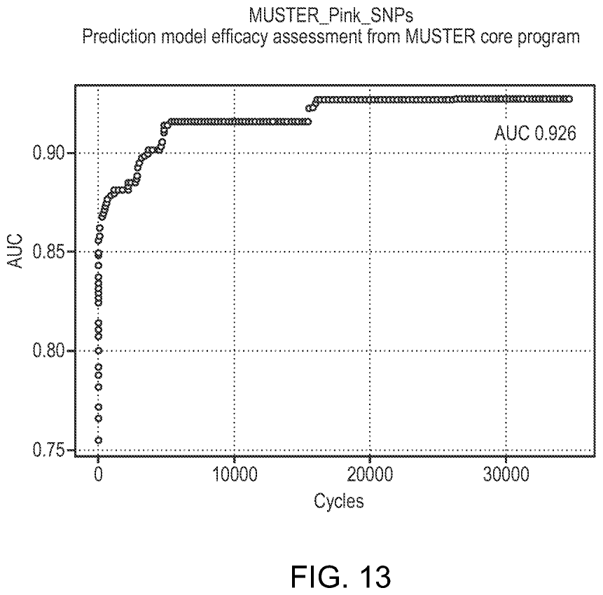

Specifically, MUSTER-Pink was used to identify a list of risk genes at the transcript level and the frequency of genomic profiles (SNPs) from reported data sets provided in a recent study from a multicenter retrospective study of patients who underwent a mastectomy at a participating translation breast cancer research consortium. The output prediction of MUSTER-Pink-RNA reached an AUC of 0.97 (FIG. 12), which was not provided by the original publication. MUSTER-Pink-SNPs also identified 30 SNPs in genomics that contribute to a risk of high cancer relapse with an AUC of 0.926 (FIG. 13), which was not built in the model in the original publication.

The major advantage of MUSTER-Pink-RNA and MUSTER-PINK-SNPs is that they provide a more accurate assessment of a risk for breast cancer relapse in patients that have undergone a mastectomy. In addition, MUSTER-Pink-SNPs also can provide genomic information to identify individuals with higher risk for preventative treatment. Thus, MUSTER-Pink-RNA and MUSTER-PINK-SNPs enhance precision treatment possibilities and provide guidance for targeted treatment development and risk management.

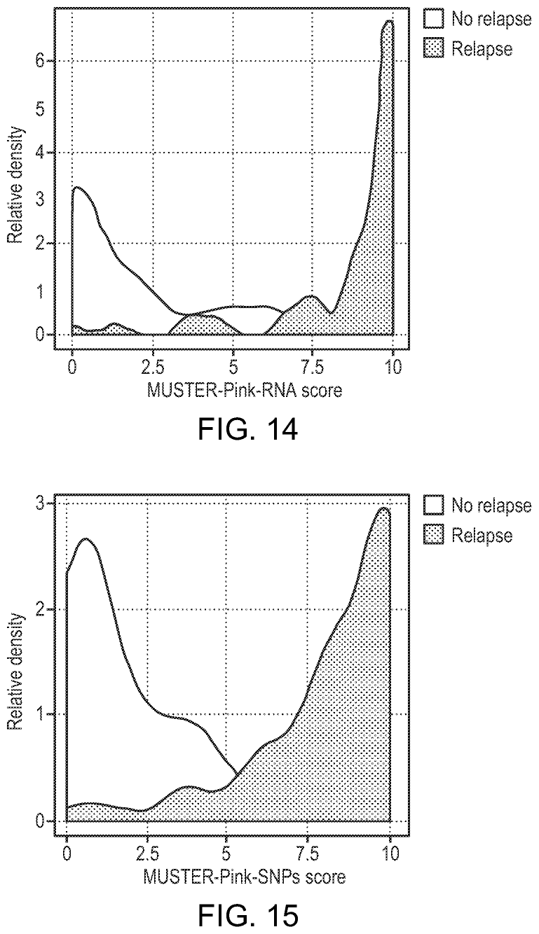

In some embodiments, MUSTER-Pink-RNA uses a set of 21 genes (Table 3) for assessment of risk of cancer relapse in patients after mastectomy. The risk of cancer relapse is provided as a risk score by the model (FIG. 14).

TABLE 3
21 Genes for MUSTER-Pink-RNA

| | | |
|---|---|---|
| DPP7 | TBCA | TRIP12 |
| RPN1 | KIF20B | IL24 |
| FAM35A | LPL | SPATA2L |
| MIR1250 | OR10A5 | ILF3 |
| ATPAF2 | ZNF616 | SAMD4A |
| FAM86HP | MLX | ARRDC2 |
| ACADVL | ACTL7A | PRLHR |

In some embodiments, MUSTER-Pink-SNPs uses a set of 30 genomic DNA SNPs (Table 4) to predict the risk of cancer recurrence in breast cancer patients after mastectomy with a scored risk outcome (FIG. 15).

TABLE 4
30 SNPs for MUSTER-Pink-SNPs

| | | |
|---|---|---|
| SNP_A_1695849, | SNP_A_1652166, | SNP_A_1697721, |
| SNP_A_1680800, | SNP_A_1704755, | SNP_A_1749217, |
| SNP_A_1696852, | SNP_A_1664806, | SNP_A_1758786, |
| SNP_A_1744363, | SNP_A_1643057, | SNP_A_1744135, |
| SNP_A_1675151, | SNP_A_1736645, | SNP_A_1665098, |
| SNP_A_1690437, | SNP_A_1716243, | SNP_A_1715095, |
| SNP_A_1672250, | SNP_A_1703233, | SNP_A_1660025, |
| SNP_A_1718357, | SNP_A_1700554, | SNP_A_1722032, |
| SNP_A_1652276, | SNP_A_1647788, | SNP_A_1703857, |
| SNP_A_1675732, | SNP_A_1750171, | SNP_A_1680220 |

Acute Myeloid Leukemia (AML)

There is no effective model in the clinic to predict the primary efficacy of acute myeloid leukemia (AML). A large portion of patients (ca. 20-30% of younger AML adults or up to 50% of older adult patients) suffer resistance to the primary treatment. To extend life expectancy, improve life quality and therapeutic strategy for all AML patients, it is a critical process to apply effective treatment in adults.

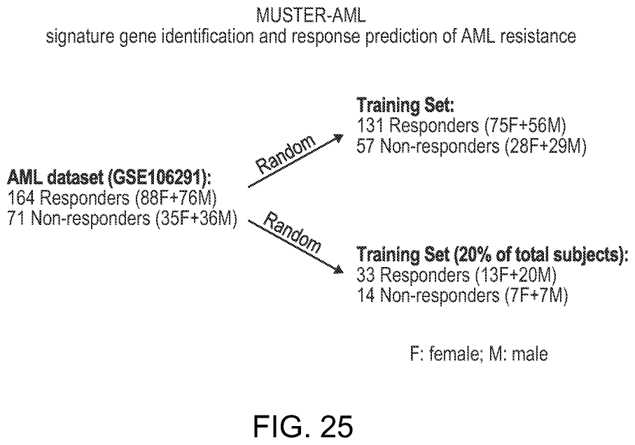

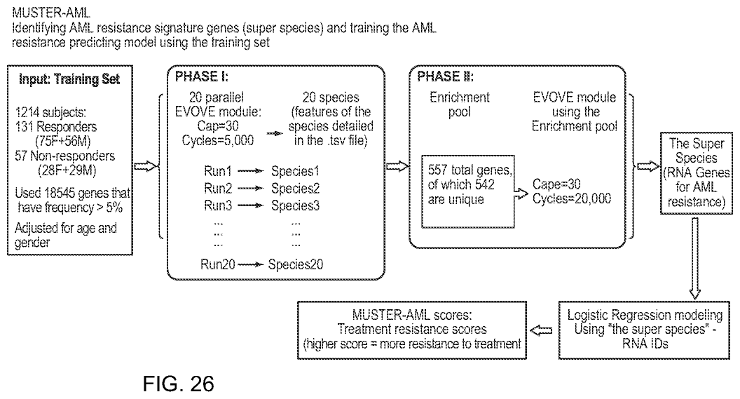

MUSTER is used to identify key genes from RNA-seq profiles in peripheral blood or bone marrow cells to predict resistance to induction treatment in AML patients (FIG. 7, FIG. 25, and FIG. 26). Specifically, MUSTER was applied to extract key feature prediction gene sets using RNA-seq profiles from peripheral blood samples of AML patients provided by the European LeukemiaNet. MUSTER-AML-RNA achieved 85% accuracy testing with p=0.0001.

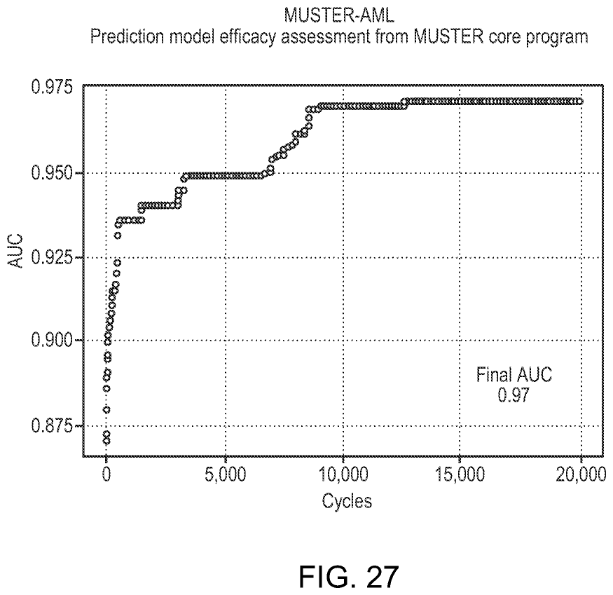

MUSTER-AML provides an efficacy of 0.97 AUC (FIG. 27).

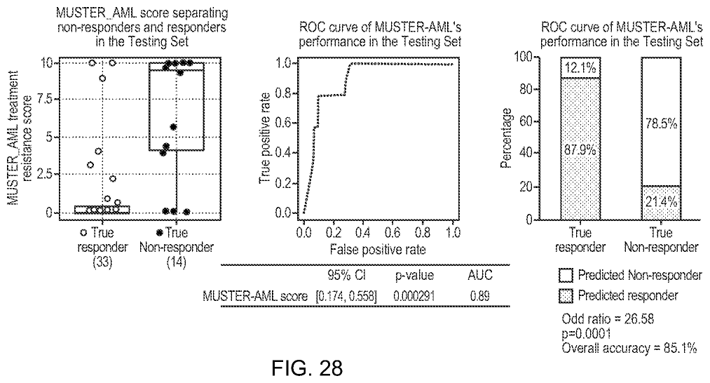

One major advantage of MUSTER-AML is that it can provide an accurate assessment if a patient is resistant to current AML treatments using gene expression patterns in blood samples (FIG. 28). Thus, the program enhances the precision treatment possibilities and provides guidance for targeted treatment development and risk management.

MUSTER-AML uses a set of 26 genes (Table 5) for assessment of the probable resistance in patients before and during their treatment.

TABLE 5
26 Genes for Muster-AML

| | | | |
|---|---|---|---|
| GAPDH, | HDGF, | LINC00152, | SLC39A3, |
| CDC123, | LOC440900, | AVPR2, | IFITM1, |
| MRPL22, | MEST, | KLHDC1, | SCARA5, |
| ADAMTS10, | BTD, | ATG5, | CROCC, |
| ZFPM1, | SDHAF1, | LRRC58, | SLC4A8, |
| PLN, | SLC35A1, | CACNG4, | ZNHIT1, |
| PRCD, | EPHX4 | | |

Post-Traumatic Stress Disorder (PTSD)

In some embodiments, MUSTER is used to identify signature genes associated with post-traumatic stress disorder (PTSD) in world trade center responders using RNA-seq profiles (FIG. 7).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of generating a feature set, based on biological signature data, used for predicting an outcome, the method comprising:
generating a representation of a mutated species from a representation of an initial species comprising biological signature data;
comparing a measured performance of the representation of the mutated species with a measured performance of the representation of the initial species based on associated outcome data;
selecting one of the representation of the mutated species and the representation of the initial species based on the results of the comparing to serve as the representation of the initial species in a next iteration of the generating and comparing; and
outputting a representation of a super species based on a final mutated species, the representation of the super species comprising a set of biological signature features predictive of the outcome.

2. The method of claim 1, wherein the measured performance is an area under a receiver operating characteristic curve determined from at least one classification threshold.

3. The method of claim 1, wherein the biological signature data comprises proteomic data, bulk transcriptome data, single-cell transcriptome data, genomic data, metabolomic data, microbiotomic data, or any combination thereof.

4. The method of claim 1, wherein the super species comprises a set of genes associated with resistance to a treatment for acute myeloid leukemia (AML).

5. The method of claim 1, wherein the super species comprises a set of genes associated with cardiovascular disease risk.

6. The method of claim 1, wherein the super species comprises a set of genes associated with cancer relapse.

7. The method of claim 1, wherein the super species comprises a set of genes associated with post-traumatic stress disorder (PTSD).

8. The method of claim 1, further comprising using the set of biological signature features to determine an outcome.

9. The method of claim 1 further comprising:
determining an outcome for test biological signature data based on the representation of the super species.

10. A method, comprising:
receiving a biological data set;
extracting an identification (ID) for each entry of the biological data set a set of IDs;
generating an initial data set from the biological data set, wherein the initial data set is a subset of the biological data set;
generating a mutated data set from the initial data set;
measuring performance of the mutated data set and the initial data set; and
selecting one of the mutated data set or the initial data set to create an output set based the performance of each respective data set.

11. The method of claim 10 wherein the performance is based on an area under a curve of a model data set.

12. The method of claim 10, wherein the biological data set comprises transcriptiomics from RNA sequence data.

13. The method of claim 10, wherein the biological data set comprises single cell transcriptomics.

14. The method of claim 10, wherein the biological data set comprises proteomics.

15. The method of claim 10, wherein the biological data set comprises single nucleotide polymorphism genomic data.

16. The method of claim 10, wherein measuring performance of the mutated data set and the initial data set comprises:
Propagating the mutated data set and the initial data set in parallel multiple times.

17. The method of claim 10, wherein the output set predicts an outcome of the biological data set.
